(19) United States
(12) Patent Application Publication
(10) Pub. No.: US 2017/0299662 A1
(43) Pub. Date: Oct. 19, 2017

(54) MAGNETIC FIELD MEASUREMENT APPARATUS AND METHOD OF CALIBRATING MAGNETIC FIELD MEASUREMENT APPARATUS

(71) Applicant: SEIKO EPSON CORPORATION, Tokyo (JP)

(72) Inventor: Kimio NAGASAKA, Hokuto-shi (JP)

(73) Assignee: SEIKO EPSON CORPORATION, Tokyo (JP)

(21) Appl. No.: 15/478,614

(22) Filed: Apr. 4, 2017

(30) Foreign Application Priority Data
Apr. 14, 2016 (JP) 2016-081355

(51) Int. Cl. G01R 33/00; A61B 5/04; G01R 33/02

(52) U.S. Cl. CPC G01R 33/0017; G01R 33/0206; A61B 5/04007; A61B 5/04008

(57) ABSTRACT

A magnetic field measurement apparatus includes a plurality of magnetic sensors, a calibration unit that estimates a magnetic field on the basis of detected vectors of the magnetic sensors, positional information of the magnetic sensors, and measured values of the magnetic sensors, and updates the detected vectors on the basis of the estimated magnetic field, and a magnetic field calculation unit that calculates a magnetic field to be measured on the basis of the measured values of the magnetic sensors and the detected vectors updated by the calibration unit.

This application claims the benefit of Japanese Patent Application No. 2016-081355, filed on Apr. 14, 2016. The content of the aforementioned application is incorporated herein by reference in its entirety.

### BACKGROUND

### 1. Technical Field

The present invention relates to a magnetic field measurement apparatus and a method of calibrating the magnetic field measurement apparatus.

### 2. Related Art

There have been known magnetic field measurement apparatuses for measuring a biomagnetic field such as a magnetic field of the heart (heart magnetic field) or a magnetic field of the brain (brain magnetic field) which is weaker than terrestrial magnetism. Since the magnetic field measurement apparatus is a non-invasive apparatus, it is possible to measure the state of an internal organ without placing burden on a test subject (living body) by the magnetic field measurement apparatus.

JP-A-7-280904 discloses a calibration method of estimating parameters of a vector position of a SQUID flux meter, a direction vector of a detected magnetic field, and the sensitivity of the magnetic field by a least squares method from a difference between a theoretical value of an output voltage of the SQUID flux meter which is calculated from a theoretical value of the magnetic field at the position of the SQUID flux meter and a measured value of the output voltage.

Incidentally, for example, in a case where a magnetic field is measured on the assumption that all detection axes of respective magnetic sensors included in a magnetic field measurement apparatus face in the same direction, there are actually variations in the directions of the detection axes, and thus there is a problem in that an error of a measured value of the magnetic field becomes large. For example, in a case where there is a difference of 1/1000=0.057 degrees between the directions of detection axes of two magnetic sensors, a measured value of the magnetic field which is obtained from outputs of the two magnetic sensors has an error of 2.6/1000=2.6 pT on the assumption that the level of magnetic noise is 2.6 nT. This error has a level exceeding a measurement resolution (for example, 1 pT) which is required for the measurement of a week magnetic field such as a heart magnetic field or a brain magnetic field, and thus desired performance is not obtained.

However, in the calibration method of JP-A-7-280904, variations in the directions of the detection axes of the SQUID flux meter are not considered, and thus it is difficult to correctly perform calibration even when the calibration method of JP-A-7-280904 is applied to a magnetic field measurement apparatus in which the directions of detection axes of magnetic sensors are not aligned.

### SUMMARY

An advantage of some aspects of the invention is to provide a magnetic field measurement apparatus capable of calculating a magnetic field to be measured with a high level of accuracy in consideration of the directions of detection axes of magnetic sensors. Another advantage of some aspects of the invention is to provide a method of calibrating the magnetic field measurement apparatus by which the magnetic field measurement apparatus can calculate a magnetic field to be measured with a high level of accuracy in consideration of the directions of detection axes of magnetic sensors.

The invention can be implemented as the following forms or application examples.

### APPLICATION EXAMPLE 1

A magnetic field measurement apparatus according to this application example includes a plurality of magnetic sensors, a calibration unit that estimates a magnetic field on the basis of detected vectors of the magnetic sensors, positional information of the magnetic sensors, and measured values of the magnetic sensors, and updates the detected vectors on the basis of the estimated magnetic field, and a magnetic field calculation unit that calculates a magnetic field to be measured on the basis of the measured values of the magnetic sensors and the detected vectors updated by the calibration unit.

According to the magnetic field measurement apparatus of this application example, the calibration unit estimates the magnetic field on the basis of the detected vectors of the magnetic sensors, the positional information of the magnetic sensors, and the measured values of the magnetic sensors, for example, in a state where the magnetic field to be measured is not measured by the magnetic sensors, and thus can update the detected vectors (information regarding the directions and gains of detection axes) of the magnetic sensors with a high level of accuracy. Therefore, according to the magnetic field measurement apparatus of the application example, the magnetic field calculation unit can calculate the magnetic field to be measured with a high level of accuracy in consideration of the directions of detection axes of the magnetic sensors by using the detected vectors updated with a high level of accuracy.

### APPLICATION EXAMPLE 2

In the magnetic field measurement apparatus according to the application example, the calibration unit may estimate the measured values of the magnetic sensors on the basis of the updated detected vectors and the estimated magnetic field, and may repeat a process of estimating the magnetic field and a process of updating the detected vectors until norms of differences between the estimated measured values of the magnetic sensors and measured values of the magnetic sensors become smaller than a threshold value.

According to the magnetic field measurement apparatus of this application example, the calibration unit can converge the detected vectors to values approximate to true values by repeatedly performing a process of estimating the magnetic field and a process of updating the detected vectors. Therefore, according to the magnetic field measurement apparatus of the application example, the magnetic field calculation unit can calculate the magnetic field to be measured with a high level of accuracy by using the detected vectors approximate to the true values.

### APPLICATION EXAMPLE 3

In the magnetic field measurement apparatus according to the application example, initial values of the detected vectors may be design values.

According to the magnetic field measurement apparatus of this application example, the calibration unit estimates the magnetic field by using the design values having small differences from the true values as the initial values of the detected vectors, and updates the detected vectors on the basis of the estimated magnetic field, and thus it is possible to update the detected vectors to values approximate to the true values.

### APPLICATION EXAMPLE 4

In the magnetic field measurement apparatus according to the application example, the calibration unit may update a detected vector of each of the magnetic sensors, on the basis of a measured value of the magnetic sensor and an estimated value of the magnetic field at a position of the magnetic sensor in the estimated magnetic field.

According to the magnetic field measurement apparatus of this application example, the calibration unit can correctly update the detected vector to a value approximate to a true value by directly calculating the detected vector of the magnetic sensor, on the basis of the measured value of the magnetic sensor and the estimated value of the magnetic field at the position of the magnetic sensor, rather than updating the detected vector by matrix calculation.

### APPLICATION EXAMPLE 5

In the magnetic field measurement apparatus according to the application example, the calibration unit may estimate the magnetic field by approximating the magnetic field by a polynomial expression with positions of the magnetic sensors as variables and calculating the polynomial expression on the basis of the detected vectors, the positional information, and the measured values of the magnetic sensors.

According to the magnetic field measurement apparatus of this application example, the calibration unit can approximate the magnetic field by the polynomial expression with positions of the magnetic sensors as variables with a high level of accuracy, and thus can estimate the magnetic field with a high level of accuracy by calculating the polynomial expression.

### APPLICATION EXAMPLE 6

In the magnetic field measurement apparatus according to the application example, the calibration unit may calculate the polynomial expression on the assumption that divergence of the magnetic field is zero.

According to the magnetic field measurement apparatus of this application example, it is possible to reduce the number of coefficients of the polynomial expression on the condition that the divergence of the magnetic field is zero, and thus the amount of calculation of the calibration unit is reduced, or the accuracy of calculation (updating) of the detected vectors is improved.

### APPLICATION EXAMPLE 7

In the magnetic field measurement apparatus according to the application example, the calibration unit may calculate the polynomial expression on the assumption that rotation of the magnetic field is zero.

According to the magnetic field measurement apparatus of this application example, it is possible to reduce the number of coefficients of the polynomial expression on the condition that the rotation of the magnetic field is zero, and thus the amount of calculation of the calibration unit is reduced, or the accuracy of calculation (updating) of the detected vectors is improved.

### APPLICATION EXAMPLE 8

A method of calibrating a magnetic field measurement apparatus according to this application example is a method of calibrating a magnetic field measurement apparatus that calculates a magnetic field to be measured on the basis of measured values of a plurality of magnetic sensors and detected vectors of the plurality of magnetic sensors, the method including acquiring the measured values of the magnetic sensors, estimating the magnetic field on the basis of the detected vectors, positional information of the magnetic sensors, and the measured values of the magnetic sensors, and updating the detected vectors on the basis of the estimated magnetic field.

According to the method of calibrating the magnetic field measurement apparatus of this application example, for example, it is possible to update the detected vectors (information regarding the directions and gains of detection axes) of the magnetic sensors with a high level of accuracy by estimating the magnetic field on the basis of the detected vectors of the magnetic sensors, the positional information of the magnetic sensors, and the measured values of the magnetic sensors, for example, in a state where the magnetic field to be measured is not measured by the magnetic sensors.

### BRIEF DESCRIPTION OF THE DRAWINGS

The invention will be described with reference to the accompanying drawings, wherein like numbers reference like elements.

FIG. 1 is a schematic side view illustrating a configuration example of a magnetic field measurement apparatus according to this embodiment.

FIG. 2 is a schematic side view of a magnetic sensor unit.

FIG. 3 is a schematic plan view of the magnetic sensor unit.

FIG. 4 is a diagram illustrating a configuration example of a processing apparatus.

FIG. 5 is a diagram illustrating a calibration method of the magnetic field measurement apparatus according to this embodiment.

FIG. 6 is a flow chart illustrating an example of a procedure in which a calibration unit of the processing apparatus performs a calibration process.

FIG. 7 is a block diagram corresponding to processes of step S3 to step S7 of FIG. 6.

FIG. 8 is a flow chart illustrating an example of a procedure of a process of updating a detected vector matrix.

FIG. 9 is a flow chart illustrating an example of a procedure of performing a magnetic field calculation process by a magnetic field calculation unit of the processing apparatus.

### DESCRIPTION OF EXEMPLARY EMBODIMENTS

Hereinafter, preferred embodiments of the invention will be described in detail with reference the accompanying drawings. Meanwhile, the embodiments described below are not unduly limited to the disclosure of the invention described in the appended claims. In addition, all the configurations described below are not necessarily essential components of the invention.

### 1. First Embodiment

### 1-1. Configuration of Magnetic Field Measurement Apparatus

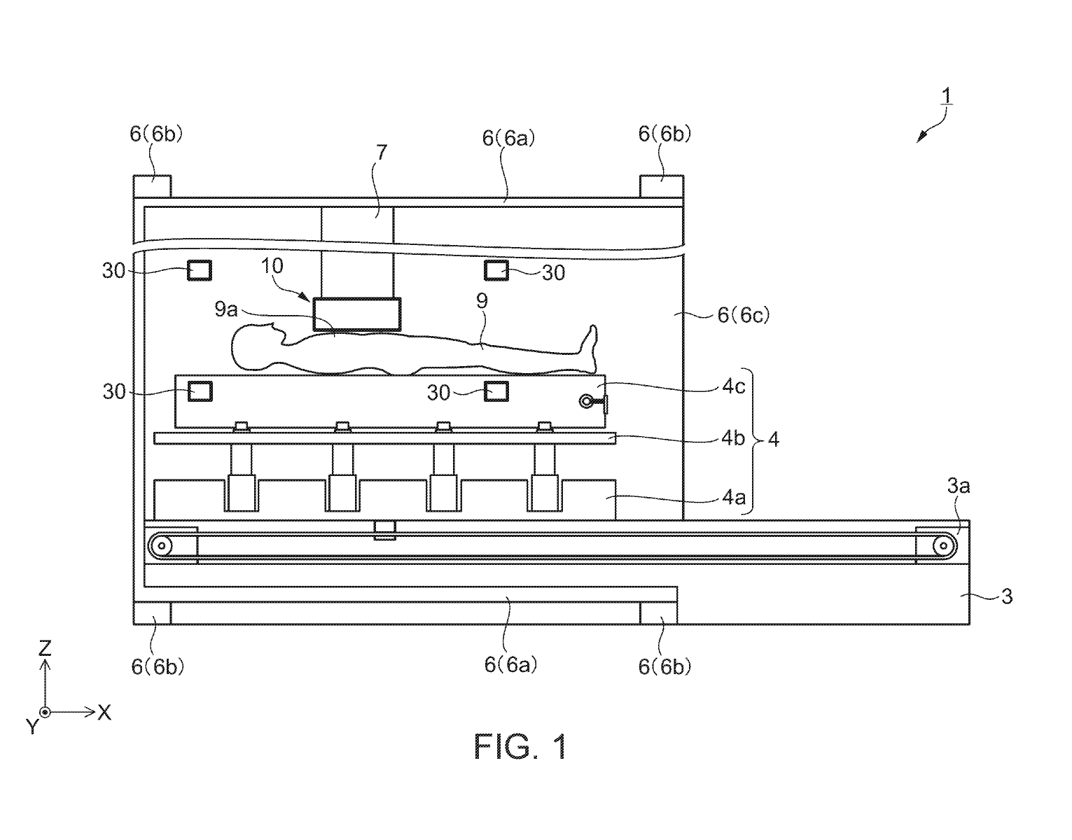

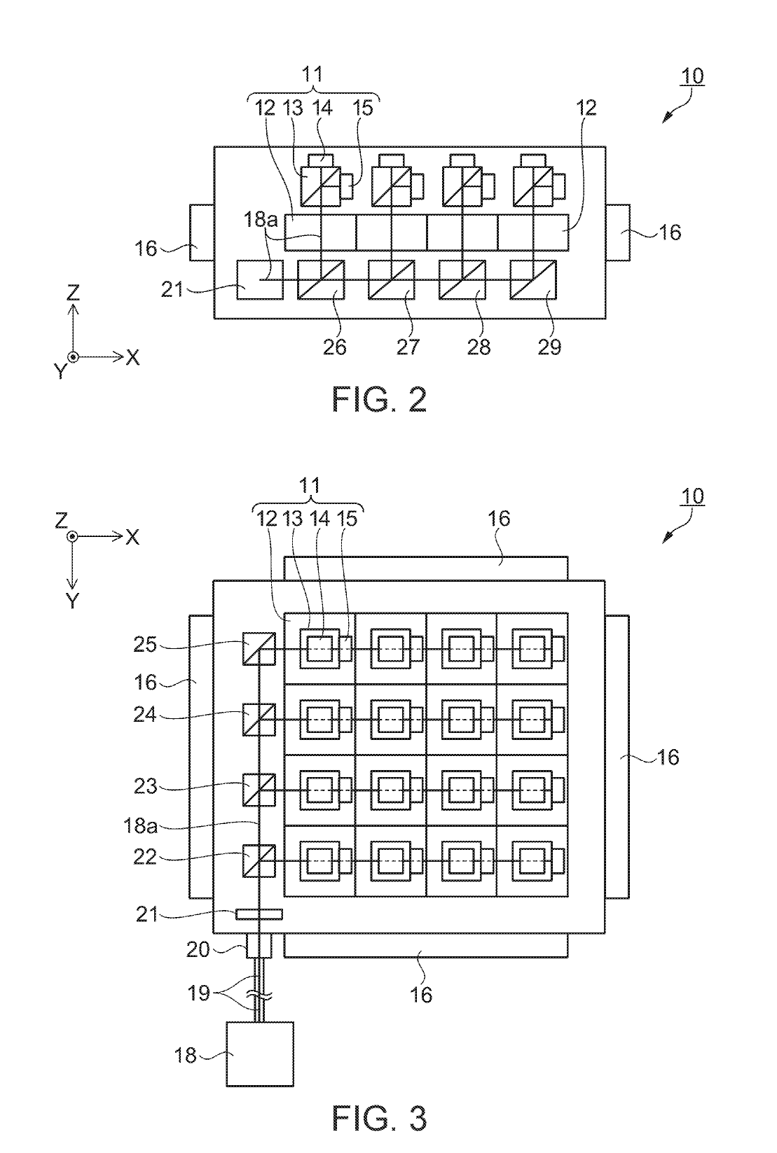

FIG. 1 is a schematic side view illustrating a configuration example of a magnetic field measurement apparatus according to this embodiment. As illustrated in FIG. 1, a magnetic field measurement apparatus 1 of this embodiment is an apparatus that measures, as a measurement object, a heart magnetic field generated from the heart of a test subject (living body) 9, or a brain magnetic field generated from the brain of the test subject (living body) 9, and the like. As illustrated in FIG. 1, the magnetic field measurement apparatus 1 includes a magnetic sensor unit 10 including a first magnetic sensor 11 (see FIGS. 2, 3, and 5) which is not shown in the drawing, a second magnetic sensor 30, a processing apparatus 2 (see FIG. 5) which is not shown in the drawing, a base 3, a table 4, and a magnetic shielding apparatus 6.

The first magnetic sensor 11 included in the magnetic sensor unit 10 is a sensor that measures a week magnetic field (a magnetic field to be measured) such as a heart magnetic field or a brain magnetic field which serves as a measurement object, and is used as a magnetocardiograph, a magnetoencephalography, or the like. The second magnetic sensor 30 is a sensor for measuring an environmental magnetic field such as an external magnetic field (magnetic noise). As the first magnetic sensor 11 and the second magnetic sensor 30, an optically pumped type magnetic sensor, a SQUID type magnetic sensor, a flux gate magnetic sensor, a MI sensor, a Hall element, or the like can be used.

The height direction (up-down direction in FIG. 1) of the magnetic field measurement apparatus 1 is set to be a Z-direction. The Z-direction is a vertical direction. Directions in which the upper surfaces of the base 3 and the table 4 extend are set to be an X-direction and a Y-direction. The X-direction and the Y-direction are horizontal directions, and are perpendicular to each other. The stature direction (right-left direction in FIG. 1) of the test subject 9 who is lying down is set to be the X-direction.

The base 3 is disposed on the bottom surface on the inner side of the magnetic shielding apparatus 6 (main body portion 6a), and extends along the X-direction (movable direction of the test subject 9) up to the outside of the main body portion 6a. The table 4 includes an X-direction table 4a, a Z-direction table 4b, and a Y-direction table 4c. The X-direction table 4a, moving along the X-direction by an X-direction linear motion mechanism 3a, is installed on the base 3. The Z-direction table 4b, which is elevated along the Z-direction by an elevation apparatus not shown in the drawing, is installed on the X-direction table 4a. The Y-direction table 4c, moving along the Y-direction on a rail by a Y-direction linear motion mechanism not shown in the drawing, is installed on the Z-direction table 4b.

The magnetic shielding apparatus 6 includes the main body portion 6a, having a square tubular shape, which includes an opening portion 6c. The inside of the main body portion 6a is hollow, and the cross-sectional shape of a surface (plane perpendicular to the X-direction in a Y-Z cross-section) which passes through the Y-direction and the Z-direction is substantially a quadrangular shape. When a heart magnetic field is measured, the test subject 9 is accommodated in the main body portion 6a in a state of lying on the table 4. The main body portion 6a extends in the X-direction, and functions as a passive magnetic shield by itself.

The magnetic sensor unit 10 and the second magnetic sensors 30 are disposed inside the main body portion 6a of the magnetic shielding apparatus 6. The magnetic shielding apparatus 6 suppresses a situation in which an external magnetic field such as terrestrial magnetism flows into a space for disposing the magnetic sensor unit 10. That is, the space for disposing the magnetic sensor unit 10 is set to be in a state of a magnetic field significantly lower than the external magnetic field by the magnetic shielding apparatus 6, and thus the influence of the external magnetic field on the magnetic sensor unit 10 is suppressed.

The base 3 protrudes in the +X-direction from the opening portion 6c of the main body portion 6a. Regarding the size of the magnetic shielding apparatus 6, for example, the length in the X-direction is approximately 200 cm, and one side of the opening portion 6c is approximately 90 cm. The test subject 9 lying on the table 4 can move on the base 3 together with the table 4 along the X-direction to enter the magnetic shielding apparatus 6 from the opening portion 6c.

The processing apparatus 2 not shown in the drawing is an apparatus that receives an electrical signal from the first magnetic sensor 11 included in the magnetic sensor unit 10 and an electrical signal from the second magnetic sensor 30 to thereby measures a magnetic field such as a heart magnetic field or a brain magnetic field. A magnetic field ora residual magnetic field which is generated by an electrical signal, generated by the processing apparatus 2, and is detected by the magnetic sensor unit 10 changes to noise. For this reason, it is preferable that the processing apparatus 2 is installed at a location apart from the opening portion 6c of the magnetic shielding apparatus 6 so that a generated magnetic field or a residual magnetic field hardly reaches the magnetic sensor unit 10.

The main body portion 6a of the magnetic shielding apparatus 6 is formed of, for example, a ferromagnetic body having a relative permeability of several thousands or more or a conductor with high conductivity. As the ferromagnetic body, Permalloy, ferrite, iron-, chromium-, or cobalt-based amorphous, or the like can be used. As the conductor with high conductivity, a conductor such as aluminum which has an effect of reducing a magnetic field by an eddy current effect can be used. Meanwhile, it is also possible to form the main body portion 6a by alternately laminating the ferromagnetic body and the conductor with high conductivity.

A correction coil (Helmholtz coil) 6b is installed at an end on the +X-direction side of the main body portion 6a and on the −X-direction side of the base 3. The correction coil 6b has a frame shape and is disposed so as to surround the main body portion 6a. The correction coil 6b is a coil for correcting an inflow magnetic field that flows into the internal space of the main body portion 6a. The inflow magnetic field indicates a magnetic field which is an external magnetic field that passes through the opening portion 6c and enters the internal space. The inflow magnetic field becomes strongest in the X-direction with respect to the opening portion 6c. The correction coil 6b generates a magnetic field so as to cancel the inflow magnetic field by a current supplied from the processing apparatus 2.

The magnetic sensor unit 10 is fixed to the ceiling of the main body portion 6a through a supporting member 7. The magnetic sensor unit 10 measures an intensity component of a magnetic field in the Z-direction. That is, the detection axis of each of the first magnetic sensors 11 included in the magnetic sensor unit 10 faces in the Z-direction. When a heart magnetic field of the test subject 9 is measured, the X-direction table 4a and the Y-direction table 4c are moved so that a chest 9a which is a measurement position in the test subject 9 is set to be at a position facing the magnetic sensor unit 10, and the Z-direction table 4b is lifted up so that the chest 9a approaches the magnetic sensor unit 10.

The plurality of second magnetic sensors 30 are disposed in the vicinity of the magnetic sensor unit 10. Each of the second magnetic sensors 30 measures components of a magnetic field in the X-direction, the Y-direction, or the Z-direction. That is, the detection axis of each of the second magnetic sensors 30 faces in the X-direction, the Y-direction, or the Z-direction.

### 1-2. Configuration of Magnetic Sensor Unit

FIGS. 2 and 3 are schematic diagrams illustrating the structure of the magnetic sensor unit 10 according to this embodiment. In detail, FIG. 2 is a schematic side view of the magnetic sensor unit 10, and FIG. 3 is a schematic plan view of the magnetic sensor unit 10.

As illustrated in FIG. 3, a laser beam 18a is supplied to the magnetic sensor unit 10 from a laser light source 18. The laser beam 18a emitted from the laser light source 18 is supplied to the magnetic sensor unit 10 via an optical fiber 19. The magnetic sensor unit 10 and the optical fiber 19 are connected to each other through an optical connector 20.

The laser light source 18 outputs the laser beam 18a having a wavelength based on an absorption line of cesium. The wavelength of the laser beam 18a is not particularly limited, but is set to, for example, a wavelength of 894 nm equivalent to a D1 line in this embodiment. The laser light source 18 is a tunable laser, and the laser beam 18a which is output from the laser light source 18 is a continuous light having a fixed amount of light.

The laser beam 18a supplied through the optical connector 20 advances in the −Y-direction to be incident on a polarizing plate 21. The laser beam 18a having passed through the polarizing plate 21 changes to linearly polarized light. The laser beam 18a is sequentially incident on a first half mirror 22, a second half mirror 23, a third half mirror 24, and a first reflective mirror 25.

The first half mirror 22, the second half mirror 23, and the third half mirror 24 reflect a portion of the laser beam 18a to advance the reflected beam in the +X-direction, and transmit a portion of the laser beam 18a to advance the transmitted beam in the −Y-direction. The first reflective mirror 25 reflects the entire incident laser beam 18a in the +X-direction. The laser beam 18a is split into four light paths by the first half mirror 22, the second half mirror 23, the third half mirror 24, and the first reflective mirror 25. The reflectances of the respective mirrors are set so that the light intensities of the laser beam 18a in the respective light paths are set to be the same light intensity.

Next, as illustrated in FIG. 2, the laser beam 18a is sequentially incident on a fourth half mirror 26, a fifth half mirror 27, a sixth half mirror 28, and a second reflective mirror 29. The fourth half mirror 26, the fifth half mirror 27, and the sixth half mirror 28 reflect a portion of the laser beam 18a to advance the reflected beam in the +Z-direction, and transmit a portion of the laser beam 18a to advance the transmitted beam in the +X-direction. The second reflective mirror 29 reflects the entire incident laser beam 18a in the +Z-direction.

One light path of the laser beam 18a is split into four light paths by the fourth half mirror 26, the fifth half mirror 27, the sixth half mirror 28, and the second reflective mirror 29. The reflectances of the respective mirrors are set so that the light intensities of the laser beam 18a in the respective light paths are set to be the same light intensity. Therefore, the laser beam 18a is divided into 16 light paths. The reflectances of the respective mirrors are set so that the light intensities of the laser beam 18a in the respective light paths are set to be the same intensity.

Here, 16 gas cells 12 of four rows by four columns are installed in the light paths of the laser beam 18a on the +Z-direction sides of the fourth half mirror 26, the fifth half mirror 27, the sixth half mirror 28, and the second reflective mirror 29. The laser beam 18a reflected by the fourth half mirror 26, the fifth half mirror 27, the sixth half mirror 28, and the second reflective mirror 29 passes through the gas cells 12. The gas cell 12 is a box having voids therein, and gas of an alkali metal is enclosed in the voids. The alkali metal is not particularly limited, and potassium, rubidium, or cesium can be used. In this embodiment, for example, cesium can be used for the alkali metal.

A polarized light separator 13 is installed on the +Z-direction side of each of the gas cells 12. The polarized light separator 13 is an element that separates the incident laser beam 18a into laser beams 18a of two polarization components perpendicular to each other. For example, a Wollaston prism or a polarization beam splitter can be used for the polarized light separator 13.

A first light detector 14 is installed on the +Z-direction side of the polarized light separator 13, and a second light detector 15 is installed on the +X-direction side of the polarized light separator 13. The laser beam 18a having passed through the polarized light separator 13 is incident on the first light detector 14, and the laser beam 18a reflected by the polarized light separator 13 is incident on the second light detector 15. Each of the first light detector 14 and the second light detector 15 outputs a current based on the light intensity of the incident laser beam 18a to the processing apparatus 2.

Since there is a possibility that the generation of a magnetic field by the first light detector 14 and the second light detector 15 affects measurement, it is preferable that the first light detector 14 and the second light detector 15 are formed of a nonmagnetic material. The magnetic sensor unit 10 includes heaters 16 which are installed on both surface in the X-direction and both surface in the Y-direction. It is preferable that the heater 16 is configured not to generate a magnetic field. For example, it is possible to use a type of heater that performs heating by making vapor or hot air pass through a flow passage. Instead of the heater, the dielectric heating of the gas cell 12 may be performed by a high frequency voltage.

The magnetic sensor unit 10 is disposed on the +Z-direction side of the test subject 9 (see FIG. 1). A magnetic vector generated by the test subject 9 enters the magnetic sensor unit 10 from the −Z-direction side. The magnetic vector passes through the fourth half mirror 26 to the second reflective mirror 29, passes through the gas cells 12, and passes through the polarized light separators 13 to come out of the magnetic sensor unit 10.

The cesium in the gas cell 12 is heated to be in a gas state. The cesium gas is irradiated with the laser beam 18a transformed into linearly polarized light, thereby exciting cesium atoms and aligning the direction of magnetic moment. When the magnetic vector passes through the gas cells 12 in this state, the magnetic moment of the cesium atoms precesses by the magnetic field of the magnetic vector. This precession is referred to as Larmor precession.

The magnitude of the Larmor precession has a positive correlation with the intensity of the magnetic field of the magnetic vector. The Larmor precession rotates a deflected surface of the laser beam 18a. The magnitude of the Larmor precession and the amount of variation in a rotation angle of the deflected surface of the laser beam 18a have a positive correlation. Therefore, the intensity of the magnetic field and the amount of variation in the rotation angle of the deflected surface of the laser beam 18a have a positive correlation.

The polarized light separator 13 separates the laser beam 18a into linearly polarized light beams of two components perpendicular to each other. The first light detector 14 and the second light detector 15 detect the intensity of the linearly polarized light beams of the two components perpendicular to each other. Thereby, the first light detector 14 and the second light detector 15 can detect the rotation angle of the deflected surface of the laser beam 18a. The processing apparatus 2 can calculate a magnetic field from a variation in the rotation angle of the deflected surface of the laser beam 18a.

The first magnetic sensor 11 is constituted by the gas cell 12, the polarized light separator 13, the first light detector 14, and the second light detector 15. The first magnetic sensor 11 is a sensor referred to as an optically pumped type magnetic sensor or an optically pumped atom magnetic sensor. The sensitivity of the first magnetic sensor is high in the Z-direction and is low in a direction perpendicular to the Z-direction. As illustrated in FIG. 3, for example, 16 first magnetic sensors 11 of four rows by four columns are disposed in the magnetic sensor unit 10. The number of first magnetic sensors 11 and the arrangement thereof in the magnetic sensor unit 10 are not particularly limited. The number of rows of the first magnetic sensors 11 may be three or less or may be five or more. Similarly, the number of columns of the first magnetic sensors 11 may be three or less or may be five or more. As the number of first magnetic sensors 11 increases, the spatial resolution can be increased.

### 1-3. Configuration of Second Magnetic Sensor

The inflow of an external magnetic field into a measurement object space having the magnetic sensor unit 10 disposed therein is suppressed by the magnetic shielding apparatus 6 (see FIG. 1), but it is difficult to completely eliminate the inflow of the external magnetic field. The second magnetic sensor 30 is a sensor for measuring an environmental magnetic field (magnetic noise) in a measurement object space in which the magnetic sensor unit 10 is disposed. Meanwhile, the second magnetic sensor 30 may detect a magnetic field to be measured (heart magnetic field) together with an environmental magnetic field (magnetic noise).

All of the detection axes of the plurality of second magnetic sensors 30 may face in the Z-direction, but it is preferable that at least two detection axes among the detection axes (detected vector kᵢ to be described later) of the plurality of second magnetic sensors 30 are perpendicular to each other. For example, the detection axis of at least one second magnetic sensor 30 may face in the Z-direction, and the detection axes of the other second magnetic sensors 30 may face in the X-direction or the Y-direction. Thereby, it is possible to estimate the distribution of a planar environmental magnetic field or the distribution of a spatial environmental magnetic field in the vicinity of the second magnetic sensors 30 with a higher level of accuracy than in a case where all of the detection axes of the second magnetic sensors 30 face in the Z-direction, thereby improving the accuracy of calibration (accuracy of calculation of a detected vector matrix K to be described later) which is performed by a method of calibrating the magnetic field measurement apparatus to be described later according to this embodiment.

Although the type of sensor used as the second magnetic sensor 30 is not limited, it is possible to use, for example, an optically pumped type magnetic sensor which is the same as the first magnetic sensor 11 mentioned above. That is, similarly to the first magnetic sensor 11, the second magnetic sensor 30 may include the cell (gas cell 12) which accommodates alkali metal atoms and on which linearly polarized light is incident, the polarized light separator 13 that separates light emitted from the cell into light in a first axis direction and light in a second axis direction, the first light detector 14 that detects the light in the first axis direction, and the second light detector 15 that detects the light in the second axis direction.

It is preferable that the cells (equivalent to the gas cells 12 of FIGS. 2 and 3) which are included in the second magnetic sensors 30 are disposed on the same plane. In this manner, it is possible to accommodate the cells of the plurality of second magnetic sensors 30 in one container (heat insulating mechanism) and keep the cells warm and to simplify a branching mechanism for a laser beam to each cell, and thus manufacturing costs of the magnetic field measurement apparatus 1 can be reduced.

### 1-4. Configuration of Processing Apparatus

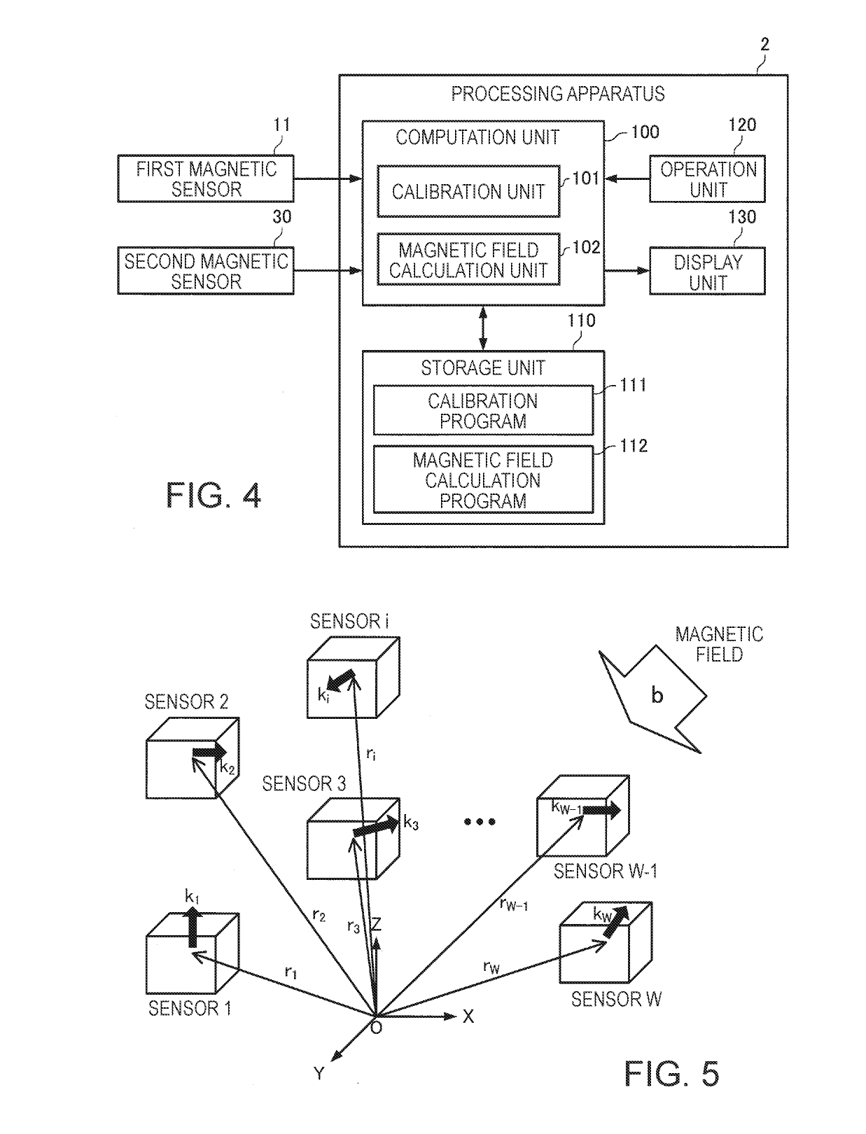

FIG. 4 is a diagram illustrating a configuration example of the processing apparatus 2. As illustrated in FIG. 4, the processing apparatus 2 is configured to include a computation unit 100, a storage unit 110, an operation unit 120, and a display unit 130.

The operation unit 120 is a unit for inputting information (various instructions such as an instruction for starting to measure a magnetic field and measurement conditions) which is necessary for a process performed by the computation unit 100, and may be any of various switches such as a button switch, a lever switch, and a dial switch, a touch panel, a keyboard, a mouse, or the like.

The display unit 130 is a unit that displays processing results of the computation unit 100 as characters, a graph, a table, an animation, or other images, and may be, for example, a liquid crystal display (LCD), an electroluminescence (EL) display, or the like.

Meanwhile, the functions of the operation unit 120 and the display unit 130 may be realized by one touch panel type display.

The storage unit 110 is a unit for storing programs, data, and the like for the computation unit 100 to perform various processes, and is constituted by any of various IC memories such as a read only memory (ROM), a flash ROM, and a random access memory (RAM), a recording medium such as a hard disk or a memory card, or the like.

Particularly, in this embodiment, the storage unit 110 stores a calibration program 111 which is read by the computation unit 100 and is used to perform a calibration process of the magnetic field measurement apparatus 1, and a magnetic field calculation program 112 for performing a process of calculating a magnetic field to be measured (magnetic field calculation process). The calibration program 111 and the magnetic field calculation program 112 may be stored in the storage unit 110 in advance, or the computation unit 100 may receive the calibration program 111 and the magnetic field calculation program 112 from a server through a network and store in the storage unit 110.

In addition, the storage unit 110 is used as a work area of the computation unit 100, and temporarily stores results of computation performed by the computation unit 100 in accordance with various programs, and the like. Further, the storage unit 110 may store data required to be stored for a long period of time among pieces of data generated by the processing of the computation unit 100.

The computation unit 100 is realized, for example, by a microprocessor such as a central processing unit (CPU), and performs the above-described calibration process, magnetic field calculation process, and the like.

In this embodiment, the computation unit 100 functions as a calibration unit 101 by executing the calibration program 111. That is, the calibration program 111 is a program for causing the processing apparatus 2 (computer) to function as the calibration unit 101 (alternatively, for causing the processing apparatus 2 to perform a calibration process). The calibration unit 101 acquires a measured value of the first magnetic sensor 11 and a measured value of the second magnetic sensor 30 to thereby perform a calibration process of the magnetic field measurement apparatus 1. Details of this calibration process will be described later.

In addition, in this embodiment, the computation unit 100 functions as a magnetic field calculation unit 102 by executing the magnetic field calculation program 112. That is, the magnetic field calculation program 112 is a program for causing the processing apparatus 2 (computer) to function as the magnetic field calculation unit 102 (alternatively, for causing the processing apparatus 2 to perform a calibration process). The magnetic field calculation unit 102 acquires a measured value of the first magnetic sensor 11 and a measured value of the second magnetic sensor 30 to thereby perform a magnetic field calculation process. Details of this magnetic field calculation process will be described later.

### 1-5. Calibration Process of Magnetic Field Measurement Apparatus

After a calibration method of the magnetic field measurement apparatus according to this embodiment is described in detail, a procedure in which the calibration unit 101 of the processing apparatus 2 performs a calibration process corresponding to the calibration method will be described.

The calibration method according to this embodiment is not limited to the first magnetic sensor 11 or the second magnetic sensor 30, and can be applied to a magnetic field measurement apparatus including any magnetic sensor. Hereinafter, in order to give a description expanded to a more general concept, the first magnetic sensor 11 and the second magnetic sensor 30 will be simply referred to as a “magnetic sensor” without making a distinction therebetween.

As illustrated in FIG. 5, it is assumed that the number of magnetic sensors W, and a detected vector kᵢ and a position vector rᵢ of each magnetic sensor i (i=1 to W) have any value. The detected vector kᵢ is a vector indicating a product of a unit vector of each magnetic sensor i in a detection axis direction and a gain of each magnetic sensor i, and the position vector rᵢ is a vector indicating a distance between a starting point O and the position of each magnetic sensor i. The calibration process of the magnetic field measurement apparatus 1 is a process of obtaining detected vectors k₁ to k_{w} of W magnetic sensors.

As illustrated in FIG. 5, a magnetic field b is applied to W magnetic sensors. It is assumed that the magnetic field b includes not only a uniform magnetic field but also a high-order gradient magnetic field. The magnetic field b for calibration may be a magnetic field which is artificially formed, or may be a natural magnetic field such as terrestrial magnetism.

Ideally, components bₓ^{(t)}, b_{y}^{(t)}, and b_{z}^{(t)} of a computational magnetic field at any point (x, y, z) at time t are made to conform to the order of distribution of a magnetic field to be measured. However, in this embodiment, it is assumed that the components are expressed by a secondary nonlinear polynomial expression of the following expression (1).

bₓ^{(t)}=aₓ₁^{(t)}+aₓ₂^{(t)}x+aₓ₃^{(t)}y+aₓ₄^{(t)}z+aₓ₅^{(t)}xy+aₓ₆^{(t)}yz+aₓ₇^{(t)}zx+aₓ₈^{(t)}x²+aₓ₉^{(t)}y²+aₓ₁₀^{(t)}x²

b_{y}^{(t)}=a_{y1}^{(t)}+a_{y2}^{(t)}x+a_{y3}^{(t)}y+a_{y4}^{(t)}z+a_{y5}^{(t)}xy+a_{y6}^{(t)}yz+a_{y7}^{(t)}zx+a_{y8}^{(t)}x²+a_{y9}^{(t)}y²+a_{y10}^{(t)}z²

b_{z}^{(t)}=a_{z1}^{(t)}+a_{z2}^{(t)}x+a_{z3}^{(t)}y+a_{z4}^{(t)}z+a_{z5}^{(t)}xy+a_{z6}^{(t)}yz+a_{z7}^{(t)}zx+a_{z8}^{(t)}x²+a_{z9}^{(t)}y²+a_{z10}^{(t)}z (1)

Here, when a calculated magnetic field value at the position of the magnetic sensor i at time t is set to be (bᵢₓ^{(t)}, b_{iy}^{(t)}, b_{iz}^{(t)}), a calculated magnetic field value vector b^{(t)} at the position of each of W magnetic sensors at the time t is expressed by the following expression (2).

\vec{b}^{(t)}=(b₁ₓ^{(t)} b_{1y}^{(t)} b_{1z}^{(t)} b₂ₓ^{(t)} b_{2y}^{(t)} . . . b_{Wx}^{(t)} b_{Wy}^{(t)} b_{Wz}^{(t)}) (2)

When calculated magnetic field value vectors b^{(l)} to b^{(T)} at times t=1 to T are integrated, a calculated magnetic field value matrix B is expressed by the following expression (3). Meanwhile, in Expression (3), tr represents the transposition of the vector.

$$B = ( \begin{matrix}{\vec{b}}^{( 1 ) tr} & {\vec{b}}^{( 2 ) tr} & … & {\vec{b}}^{( T ) tr}\end{matrix} ) = ( \begin{matrix}{b}_{1 x}^{( 1 )} & {b}_{1 x}^{( 2 )} & {b}_{1 x}^{( 3 )} & … & {b}_{1 x}^{( T )} \\ {b}_{1 y}^{( 1 )} & {b}_{1 y}^{( 2 )} & {b}_{1 y}^{( 3 )} & … & {b}_{1 y}^{( T )} \\ {b}_{1 z}^{( 1 )} & {b}_{1 z}^{( 2 )} & {b}_{1 z}^{( 3 )} & … & {b}_{1 z}^{( T )} \\ {b}_{2 x}^{( 1 )} & {b}_{2 x}^{( 2 )} & {b}_{2 x}^{( 3 )} & … & {b}_{2 x}^{( T )} \\ {b}_{2 y}^{( 1 )} & {b}_{2 y}^{( 2 )} & {b}_{2 y}^{( 3 )} & … & {b}_{2 y}^{( T )} \\ ⋮ & ⋮ & ⋮ & ⋮ & ⋮ \\ {b}_{Wx}^{( 1 )} & {b}_{Wx}^{( 2 )} & {b}_{Wx}^{( 3 )} & … & {b}_{Wx}^{( T )} \\ {b}_{Wy}^{( 1 )} & {b}_{Wy}^{( 2 )} & {b}_{Wy}^{( 3 )} & … & {b}_{Wy}^{( T )} \\ {b}_{Wz}^{( 1 )} & {b}_{Wz}^{( 2 )} & {b}_{Wz}^{( 3 )} & … & {b}_{Wz}^{( T )}\end{matrix} ) \qquad ( 3 )$$

Next, as shown in the following expression (4), a set of coefficients of the polynomial expression (1) is represented by a 30-dimensional column vector a^{(t)}. Meanwhile, in Expression (4), tr represents the transposition of the vector.

\vec{a}^{(t)}=(aₓ₁^{(t)} aₓ₂^{(t)} . . . aₓ₉^{(t)} aₓ₁₀^{(t)} a_{y1}^{(t)} a_{y2}^{(t)} . . . a_{y9}^{(t)} a_{y10}^{(t)} . . . a_{z1}^{(t)} a_{z2}^{(t)} . . . a_{z9}^{(t)} a_{z10}^{(t)})^{tr} (4)

Since it is assumed that the vector a^{(t)} varies in time series, a polynomial expression coefficient matrix A obtained by integrating vectors a^{(l)} to a^{(T)} at times t=1 to T is defined as the following expression (5). Meanwhile, in Expression (5), tr represents the transposition of the vector.

$$A = ( \begin{matrix}{\vec{a}}^{( 1 ) tr} & {\vec{a}}^{( 2 ) tr} & {\vec{a}}^{( 3 ) tr} & … & {\vec{a}}^{( T ) tr}\end{matrix} ) = ( \begin{matrix}{a}_{x 1}^{( 1 )} & {a}_{x 1}^{( 2 )} & {a}_{x 1}^{( 3 )} & … & {a}_{x 1}^{( T )} \\ {a}_{x 2}^{( 1 )} & {a}_{x 2}^{( 2 )} & {a}_{x 2}^{( 3 )} & … & {a}_{x 2}^{( T )} \\ ⋮ & ⋮ & ⋮ & ⋮ & ⋮ \\ {a}_{x 9}^{( 1 )} & {a}_{x 9}^{( 2 )} & {a}_{x 9}^{( 3 )} & … & {a}_{x 9}^{( T )} \\ {a}_{x 10}^{( 1 )} & {a}_{x 10}^{( 2 )} & {a}_{x 10}^{( 3 )} & … & {a}_{x 10}^{( T )} \\ {a}_{y 1}^{( 1 )} & {a}_{y 1}^{( 2 )} & {a}_{y 1}^{( 3 )} & … & {a}_{y 1}^{( T )} \\ {a}_{y 2}^{( 1 )} & {a}_{y 2}^{( 2 )} & {a}_{y 2}^{( 3 )} & … & {a}_{y 2}^{( T )} \\ ⋮ & ⋮ & ⋮ & ⋮ & ⋮ \\ {a}_{y 9}^{( 1 )} & {a}_{y 9}^{( 2 )} & {a}_{y 9}^{( 3 )} & … & {a}_{y 9}^{( T )} \\ {a}_{y 10}^{( 1 )} & {a}_{y 10}^{( 2 )} & {a}_{y 10}^{( 3 )} & … & {a}_{y 10}^{( T )} \\ {a}_{z 1}^{( 1 )} & {a}_{z 1}^{( 2 )} & {a}_{z 1}^{( 3 )} & … & {a}_{z 1}^{( T )} \\ {a}_{z 2}^{( 1 )} & {a}_{z 2}^{( 2 )} & {a}_{z 2}^{( 3 )} & … & {a}_{z 2}^{( T )} \\ ⋮ & ⋮ & ⋮ & ⋮ & ⋮ \\ {a}_{z 9}^{( 1 )} & {a}_{z 9}^{( 2 )} & {a}_{z 9}^{( 3 )} & … & {a}_{z 9}^{( T )} \\ {a}_{z 10}^{( 1 )} & {a}_{z 10}^{( 2 )} & {a}_{z 10}^{( 3 )} & … & {a}_{z 10}^{( T )}\end{matrix} ) \qquad ( 5 )$$

A positional information matrix P of 3 W×30 is defined as the following expression (6) on the basis of the defined secondary nonlinear polynomial expression (1).

$$P = ( \begin{matrix}1 & {x}_{1} & ⋯ & {y}_{1}^{2} & {z}_{1}^{2} & 0 & 0 & ⋯ & 0 & 0 & 0 & 0 & ⋯ & 0 & 0 \\ 0 & 0 & … & 0 & 0 & 1 & {x}_{1} & … & {y}_{1}^{2} & {z}_{1}^{2} & 0 & 0 & … & 0 & 0 \\ 0 & 0 & … & 0 & 0 & 0 & 0 & … & 0 & 0 & 1 & {x}_{1} & … & {y}_{1}^{2} & {z}_{1}^{2} \\ 1 & {x}_{2} & … & {y}_{2}^{2} & {z}_{2}^{2} & 0 & 0 & … & 0 & 0 & 0 & 0 & … & 0 & 0 \\ 0 & 0 & … & 0 & 0 & 1 & {x}_{2} & … & {y}_{2}^{2} & {z}_{2}^{2} & 0 & 0 & … & 0 & 0 \\ 0 & 0 & … & 0 & 0 & 0 & 0 & … & 0 & 0 & 1 & {x}_{2} & … & {y}_{2}^{2} & {z}_{2}^{2} \\ 1 & {x}_{3} & … & {y}_{3}^{2} & {z}_{3}^{2} & 0 & 0 & … & 0 & 0 & 0 & 0 & … & 0 & 0 \\ ⋮ & ⋮ & ⋮ & ⋮ & ⋮ & ⋮ & ⋮ & ⋮ & ⋮ & ⋮ & ⋮ & ⋮ & ⋮ & ⋮ & ⋮ \\ 1 & {x}_{W} & … & {y}_{W}^{2} & {z}_{W}^{2} & 0 & 0 & … & 0 & 0 & 0 & 0 & … & 0 & 0 \\ 0 & 0 & … & 0 & 0 & 1 & {x}_{W} & … & {y}_{W}^{2} & {z}_{W}^{2} & 0 & 0 & … & 0 & 0 \\ 0 & 0 & … & 0 & 0 & 0 & 0 & … & 0 & 0 & 1 & {x}_{W} & … & {y}_{W}^{2} & {z}_{W}^{2}\end{matrix} ) \qquad ( 6 )$$

Here, the following relational expression (7) is established. That is, the positional information matrix P is a matrix for converting the position vectors r₁ to r_{w} of W magnetic sensors into the calculated magnetic field value matrix B.

B=PA (7)

In addition, a gain matrix G including gains (equivalent to sensitivities) g₁ to g_{w} of W magnetic sensors as elements is defined as the following expression (8). The gain matrix G is a square matrix of W×W.

$$G = ( \begin{matrix}{g}_{1} & 0 & … & 0 \\ ⋮ & ⋮ & ⋮ & ⋮ \\ 0 & {g}_{2} & … & 0 \\ 0 & 0 & … & {g}_{W}\end{matrix} ) \qquad ( 8 )$$

In addition, a detection axis orientation of the magnetic sensor i is represented by a unit vector (sᵢₓ, s_{iy}, s_{iz}) on an XYZ orthogonal coordinate system, and a detection axis matrix S of W×3W which is obtained by integrating the unit vectors of the detection axis orientations of W magnetic sensors is defined as the following expression (9). Meanwhile, the relation of sᵢₓ²+s_{iy}²+s_{iz}²=1 is established.

$$S = ( \begin{matrix}{s}_{1 x} & {s}_{1 y} & {s}_{1 z} & 0 & 0 & … & 0 & 0 & 0 \\ 0 & 0 & 0 & {s}_{2 x} & {s}_{2 y} & … & 0 & 0 & 0 \\ ⋮ & ⋮ & ⋮ & ⋮ & ⋮ & ⋮ & ⋮ & ⋮ & ⋮ \\ 0 & 0 & 0 & 0 & 0 & … & {s}_{Wx} & {s}_{Wy} & {s}_{Wz}\end{matrix} ) \qquad ( 9 )$$

As shown in the following expression (10), a product of the gain matrix G and the detection axis matrix S is set to be a detected vector matrix K. Here, the detected vector kᵢ of the magnetic sensor i is (gᵢsᵢₓ, gᵢs_{iy}, gᵢs_{iz}), and the detected vector matrix K includes the detected vectors k₁ to k_{w} of W magnetic sensors as elements.

$$GS = ( \begin{matrix}{g}_{1} {s}_{1 x} & {g}_{1} {s}_{1 y} & {g}_{1} {s}_{1 z} & 0 & 0 & … & 0 & 0 & 0 \\ 0 & 0 & 0 & {g}_{2} {s}_{2 x} & {g}_{2} {s}_{2 y} & … & 0 & 0 & 0 \\ ⋮ & ⋮ & ⋮ & ⋮ & ⋮ & ⋮ & ⋮ & ⋮ & ⋮ \\ 0 & 0 & 0 & 0 & 0 & … & {g}_{W} {s}_{Wx} & {g}_{W} {s}_{Wy} & {g}_{W} {s}_{Wz}\end{matrix} ) = K \qquad ( 10 )$$

In addition, a computational observation value (estimated value) of the magnetic sensor i at time t is represented by a calculated observation value l₁^{(t)}, and a calculated observation value vector l^{(t)}, obtained by integrating calculated observation values l₁^{(t)}, to l_{w}^{(t)}, of W magnetic sensors at time t is defined as the following expression (11). Meanwhile, in Expression (11), tr represents the transposition of the vector.

\vec{l}^{(t)}′=(l₁^{(t)}′l₂^{(t)}′. . . l_{w}^{(t)}′)^{tr} (11)

At this time, a relational expression of the following expression (12) is established between a calculated observation value matrix L′ obtained by integrating calculated observation value vectors l⁽¹⁾, to l^{(T)}, at times t=1 to T, the detected vector matrix K, the positional information matrix P, and the polynomial expression coefficient matrix A. In Expression (12), the relation of D=KP is established.

L′=KPA=DA (12)

Similarly, an actual observation value (actual measurement value) of the magnetic sensor i at time t is represented by a sensor observation value lᵢ^{(t)}, and a sensor observation value vector l^{(t)} obtained by integrating sensor observation values l₁^{(t)} to l_{w}^{(t)} of W magnetic sensors at time t is defined as the following expression (13). Meanwhile, in Expression (13), tr represents the transposition of the vector.

\vec{l}^{(t)}=(l₁^{(t)} l₂^{(t)} . . . l_{W}^{(t)})^{tr} (13)

As shown in the following expression (14), a difference between the sensor observation value vector l^{(t)} and the calculated observation value vector l^{(t)}′ is set to be an observation value error vector v^{(t)}.

\vec{v}^{(t)}=\vec{l}^{(t)}−\vec{l}^{(t)}′ (14)

In addition, when a matrix obtained by integrating observation value error vectors v⁽¹⁾ to v^{(T)} at times t=1 to T is set to be an observation value error matrix V, the observation value error matrix V is represented by a difference between a sensor observation value matrix L obtained by integrating sensor observation value vectors l⁽¹⁾ to l^{(T)} at times t=1 to T and the calculated observation value matrix L′ as shown in the following expression (15).

V=L−L′ (15)

When an optimization problem for obtaining the detected vector matrix K for minimizing a norm ∥V∥ of the observation value error matrix V defined as the following expression (16) and the polynomial expression coefficient matrix A is solved, the detected vectors k₁ to k_{w} of W magnetic sensors are obtained. However, the convergence of the solution of the optimization problem may require a long time, and thus it is realistic to obtain the detected vectors k₁ to k_{w} when the norm ∥V∥ of the observation value error matrix V becomes smaller than an allowable value ε.

∥V∥=(Σₜ₌₁^{T}Σᵢ₌₁^{W}|vᵢ^{(t)}|²)^{1/2}=(Σₜ₌₁^{T}∥\vec{v}^{(t)}∥²)^{1/2} (16)

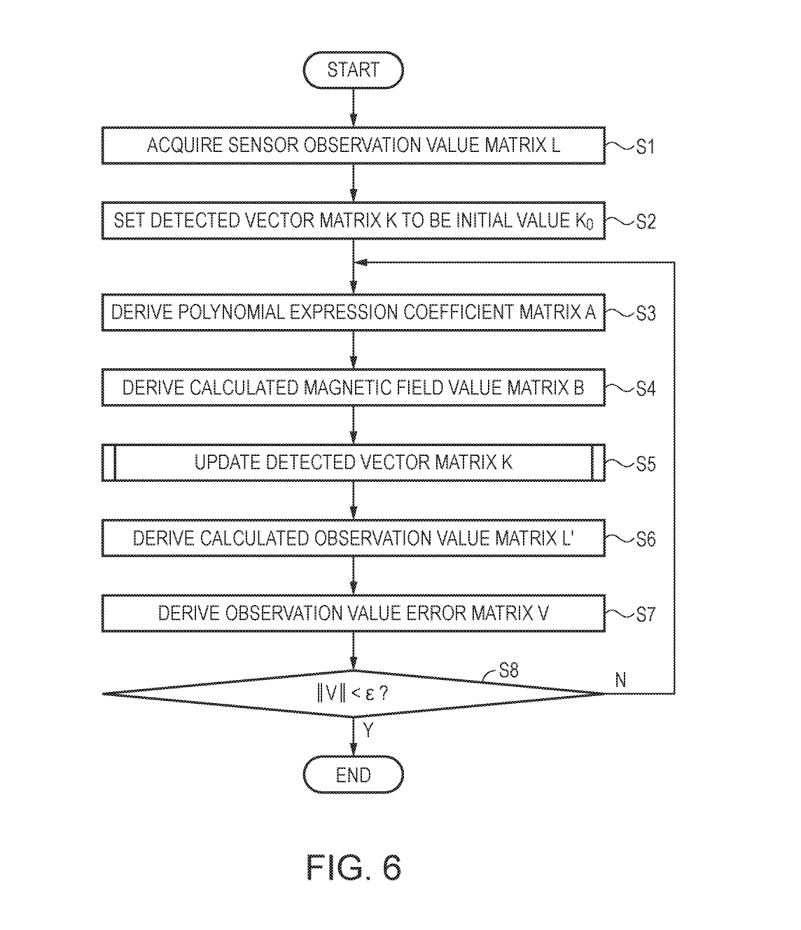

FIG. 6 is a flow chart illustrating an example of a procedure in which the calibration unit 101 (see FIG. 4) of the processing apparatus 2 performs a calibration process corresponding to the above-described calibration method of the magnetic field measurement apparatus 1. Meanwhile, the calibration process of FIG. 6 is performed in a state where the test subject (living body) 9 is not lying on the table 4 (a state where there is no influence of a heart magnetic field or a brain magnetic field from the test subject (living body) 9).

In the example of FIG. 6, first, the calibration unit 101 acquires the sensor observation value matrix L (step S1). Specifically, the calibration unit 101 acquires measured values of the respective first magnetic sensors 11 and measured values of the respective second magnetic sensors 30 at t=1 to T, and acquires the sensor observation value matrix L obtained by integrating these measured values as sensor observation value vectors l⁽¹⁾ to l^{(T)}.

Next, the calibration unit 101 sets the detected vector matrix K to be an initial value K₀ (step S2). In order to converge the detected vector matrix K to a value approximate to a true value by the processes of steps S3 to S8 to be described later, it is preferable that the initial value K₀ is a value having a small difference from the true value and may be, for example, a design value (value estimated from the arrangement of the first magnetic sensors 11 and the arrangement of the second magnetic sensors). Meanwhile, the values of respective elements of the initial value K₀ are stored in the storage unit 110 in advance.

Next, the calibration unit 101 derives the polynomial expression coefficient matrix A (step S3). Specifically, the calibration unit 101 derives the polynomial expression coefficient matrix A from the sensor observation value matrix L acquired in step S1, the detected vector matrix K (initial value K₀ which is set in step S2), and the positional information matrix P by the following expression (17). Meanwhile, the values of respective elements of the positional information matrix P are stored in the storage unit 110 in advance.

A=(KP)⁺L=D⁺L (17)

In Expression (17), (KP)⁺ is a pseudo inverse matrix of KP, and D⁺ is a pseudo inverse matrix of D(=KP). A pseudo inverse matrix D⁺(=(KP)⁺) is defined as the following expression (18). Meanwhile, in Expression (18), T represents the transposition of the matrix.

D⁺=(D^{T}D)⁻¹D^{T} (18)

Next, the calibration unit 101 derives the calculated magnetic field value matrix B (step S4). Specifically, the calibration unit 101 derives the calculated magnetic field value matrix B from the polynomial expression coefficient matrix A derived in step S3 and the positional information matrix P by Expression (7).

Next, the calibration unit 101 updates the detected vector matrix K (step S5). Specifically, the calibration unit 101 derives a matrix corresponding to a matrix product of the sensor observation value matrix L acquired in step S1 and a pseudo inverse matrix B⁺ of the calculated magnetic field value matrix B derived in step S4 and updates the detected vector matrix K. However, actually, the detected vector matrix K may not be correctly derived even when the matrix product of the sensor observation value matrix L and the pseudo inverse matrix B⁺ is calculated. Accordingly, in this embodiment, the detected vector matrix K is updated by deriving the detected vectors k₁ to k_{w} which are elements of the detected vector matrix K from sensor observation value vectors l₁ to l_{w} which are elements of the sensor observation value matrix L and calculated magnetic field value matrices b₁ to b_{w} which are elements of the calculated magnetic field value matrix B. Details of this process of updating the detected vector matrix K will be described later.

Next, the calibration unit 101 derives the calculated observation value matrix L′ (step S6). Specifically, the calibration unit 101 derives the calculated observation value matrix L′ from the calculated magnetic field value matrix B derived in step S4 and the detected vector matrix K updated in step S5 by the following expression (19).

L′=KB (19)

Next, the calibration unit 101 derives the observation value error matrix V (step S7). Specifically, the calibration unit 101 derives the observation value error matrix V from the sensor observation value matrix L acquired in step S1 and the calculated observation value matrix L′ derived in step S3 by Expression (15).

Next, the calibration unit 101 determines whether or not the norm ∥V∥ of the observation value error matrix V is smaller than the allowable value ε (step S8). Specifically, the calibration unit 101 calculates the norm ∥V∥ from the observation value error matrix V derived in step S3 by Expression (16), and compares the calculated value with the allowable value ε.

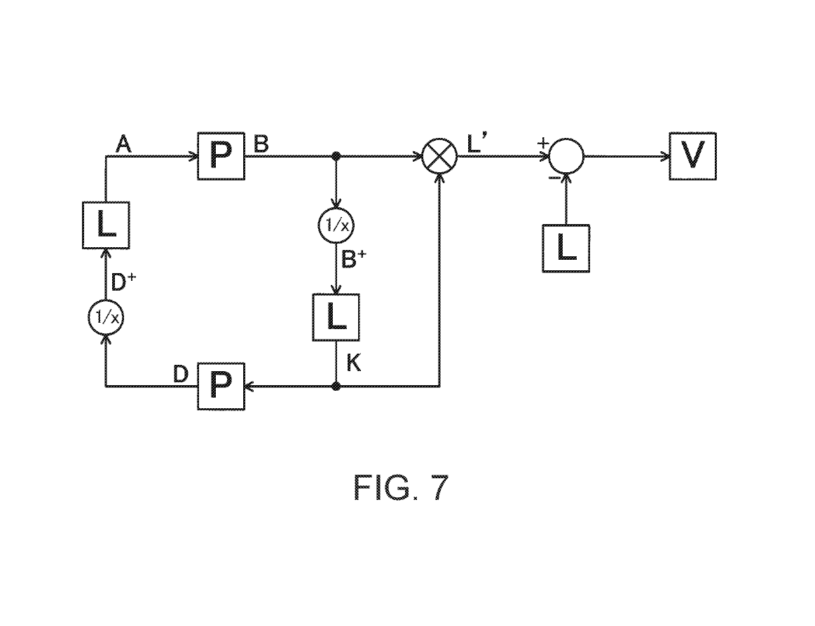

In a case where the norm ∥V∥ is equal to or greater than the allowable value ε (N in step S8), the calibration unit 101 performs the process of step S3 and the subsequent processes again. When the norm ∥V∥ becomes smaller than the allowable value ε (Y in step S8), the calibration unit terminates the calibration process. Meanwhile, FIG. 7 is a block diagram corresponding to the processes of steps S3 to S7 of FIG. 6.

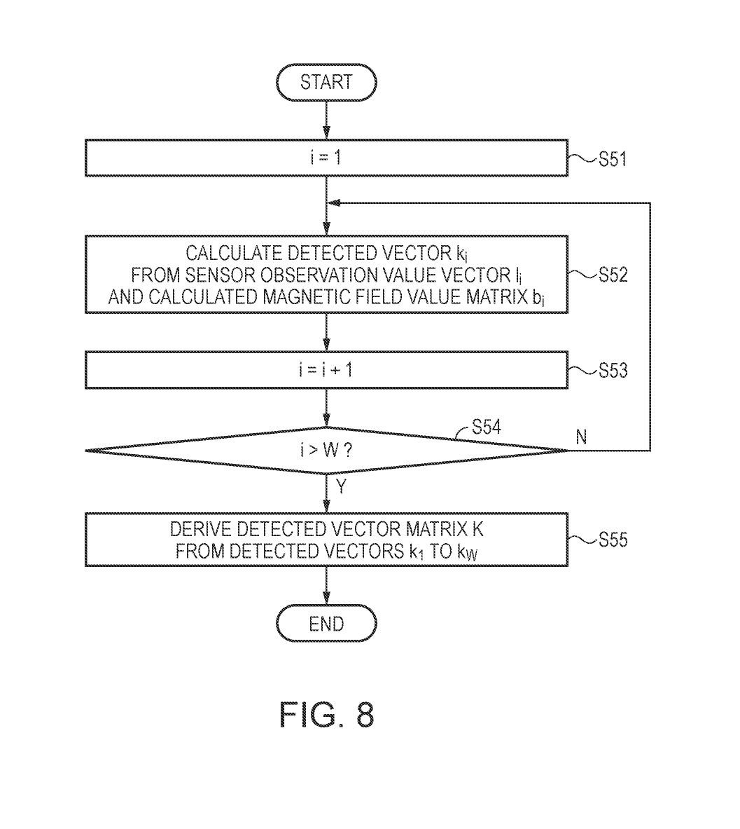

FIG. 8 is a flow chart illustrating an example of a procedure of a process of updating the detected vector matrix K (the process of step S5 of FIG. 6).

In the example of FIG. 8, first, the calibration unit 101 initializes a variable i to 1 (step S51).

Next, the calibration unit 101 calculates a detected vector kᵢ from a sensor observation value vector lᵢ and a calculated magnetic field value matrix bᵢ (step S52). Specifically, the calibration unit 101 calculates the detected vector kᵢ from the sensor observation value vector lᵢ and the calculated magnetic field value matrix bᵢ by the following expression (20). Meanwhile, in Expression (20), T represents the transposition of the vector.

\vec{k}ₗ=\vec{l}ₗbₗ^{T}(bₗbₗ^{T})⁻¹ (20)

Here, the sensor observation value vector lᵢ is defined as a vector obtained by integrating sensor observation values lᵢ⁽¹⁾ to lᵢ^{(T)} at times t=1 to T on the basis of a magnetic sensor i as shown in the following expression (21), and the sensor observation value matrix L is a matrix obtained by integrating sensor observation value vectors l₁ to l_{w} as in Expression (22).

$$\begin{matrix}{\vec{l}}_{i} = ( \begin{matrix}{l}_{i}^{( 1 )} & {l}_{i}^{( 2 )} & … & {l}_{i}^{( T )}\end{matrix} ) & ( 21 ) \\ L = ( \begin{matrix}{l}_{1}^{( 1 )} & {l}_{1}^{( 2 )} & … & {l}_{1}^{( T )} \\ {l}_{2}^{( 1 )} & {l}_{2}^{( 2 )} & … & {l}_{2}^{( T )} \\ ⋮ & ⋮ & ⋮ & ⋮ \\ \begin{matrix}{l}_{W}^{( 1 )} & \end{matrix} & {l}_{W}^{( 2 )} & … & {l}_{W}^{( T )}\end{matrix} ) = ( \begin{matrix}{\vec{l}}_{1} \\ {\vec{l}}_{2} \\ ⋮ \\ {\vec{l}}_{W}\end{matrix} ) & ( 22 )\end{matrix}$$

In addition, the calculated magnetic field value matrix bᵢ is defined as a vector obtained by integrating calculated magnetic field values at the positions of the magnetic sensors i at times t=1 to T as shown in the following expression (23), and the calculated magnetic field value matrix B is a matrix obtained by integrating the calculated magnetic field value matrices b₁ to b_{w} as in Expression (24).

$$\begin{matrix}{b}_{i} = ( \begin{matrix}{b}_{ix}^{( 1 )} & {b}_{ix}^{( 2 )} & {b}_{ix}^{( 3 )} & … & {b}_{ix}^{( T )} \\ {b}_{iy}^{( 1 )} & {b}_{iy}^{( 2 )} & {b}_{iy}^{( 3 )} & … & {b}_{iy}^{( T )} \\ {b}_{iz}^{( 1 )} & {b}_{iz}^{( 2 )} & {b}_{iz}^{( 3 )} & … & {b}_{iz}^{( T )}\end{matrix} ) & ( 23 ) \\ B = ( \begin{matrix}{b}_{1 x}^{( 1 )} & {b}_{1 x}^{( 2 )} & {b}_{1 x}^{( 3 )} & … & {b}_{1 x}^{( T )} \\ {b}_{1 y}^{( 1 )} & {b}_{1 y}^{( 2 )} & {b}_{1 y}^{( 3 )} & … & {b}_{1 y}^{( T )} \\ {b}_{1 z}^{( 1 )} & {b}_{1 z}^{( 2 )} & {b}_{1 z}^{( 3 )} & … & {b}_{1 z}^{( T )} \\ {b}_{2 x}^{( 1 )} & {b}_{2 x}^{( 2 )} & {b}_{2 x}^{( 3 )} & … & {b}_{2 x}^{( T )} \\ {b}_{2 y}^{( 1 )} & {b}_{2 y}^{( 2 )} & {b}_{2 y}^{( 3 )} & … & {b}_{2 y}^{( T )} \\ {b}_{2 z}^{( 1 )} & {b}_{2 z}^{( 2 )} & {b}_{2 z}^{( 3 )} & … & {b}_{2 z}^{( T )} \\ ⋮ & ⋮ & ⋮ & ⋮ & ⋮ \\ {b}_{Wx}^{( 1 )} & {b}_{Wx}^{( 2 )} & {b}_{Wx}^{( 3 )} & … & {b}_{Wx}^{( T )} \\ {b}_{Wy}^{( 1 )} & {b}_{Wy}^{( 2 )} & {b}_{Wy}^{( 3 )} & … & {b}_{Wy}^{( T )} \\ {b}_{Wz}^{( 1 )} & {b}_{Wz}^{( 2 )} & {b}_{Wz}^{( 3 )} & … & {b}_{Wz}^{( T )}\end{matrix} ) = ( \begin{matrix}{b}_{1} \\ {b}_{2} \\ ⋮ \\ {b}_{W}\end{matrix} ) & ( 24 )\end{matrix}$$

Next, the calibration unit 101 increments the variable i by 1 (step S53).

Next, the calibration unit 101 determines whether or not the variable i is greater than W (step S54). That is, the calibration unit 101 determines whether or not the calculation of each of the detected vectors k₁ to k_{w} has been terminated.

In a case where the variable i is equal to or less than W (N in step S54), the calibration unit 101 performs the process of step S52 and the subsequent processes again. When the variable i becomes larger than W (Y in step S54), the calibration unit derives a detected vector matrix K from the detected vectors k₁ to k_{w}, and terminates the process of updating the detected vector matrix K. As described above, the detected vector kᵢ is (gᵢsᵢₓ, gᵢs_{iy}, gᵢs_{iz}), and the calibration unit 101 derives the detected vector matrix K by integrating the detected vectors k₁ to k_{w}, as in Expression (10) (step S55).

### 1-6. Magnetic Field Measurement Process

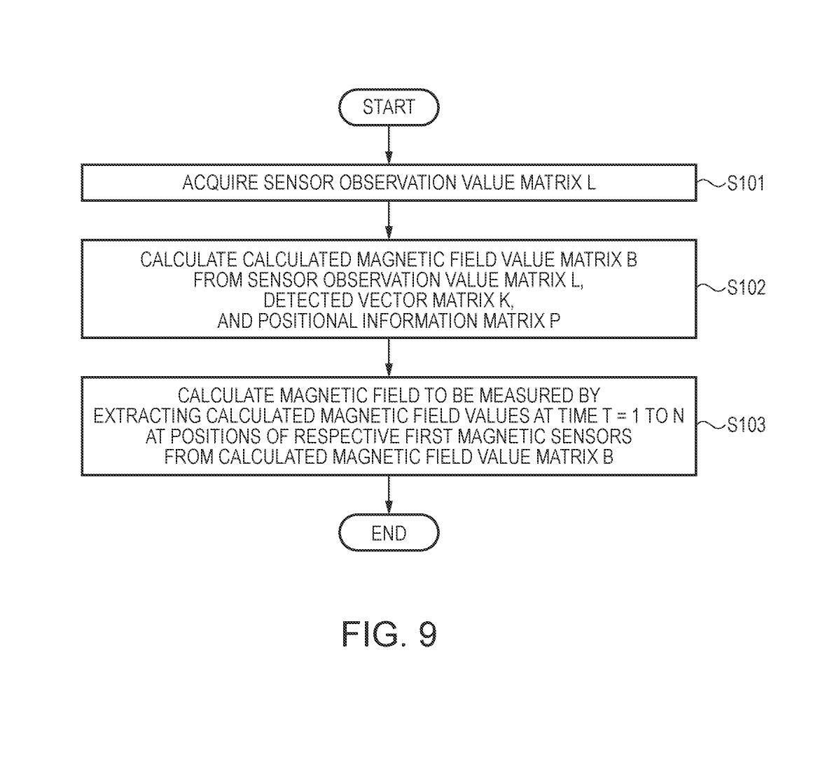

FIG. 9 is a flow chart illustrating an example of a procedure of performing a magnetic field calculation process by the magnetic field calculation unit 102 (see FIG. 4) of the processing apparatus 2. Meanwhile, the magnetic field calculation process of FIG. 9 is performed in a state where the test subject (living body) 9 is lying on the table 4 (a state where a heart magnetic field or a brain magnetic field can be measured from the test subject (living body) 9). In addition, it is assumed that a calibration process of which the procedure is illustrated as an example in FIG. 6 is performed prior to the magnetic field calculation process of FIG. 9. That is, in the calibration process, it is assumed that the detected vector matrix K is updated (calculated) on the basis of the sensor observation value matrix L, and the values of the elements of the detected vector matrix K are stored in the storage unit 110.

In the example of FIG. 9, first, the magnetic field calculation unit 102 acquires the sensor observation value matrix L (step S101). Specifically, the magnetic field calculation unit 102 acquires measured values of the respective first magnetic sensors 11 and measured values of the respective second magnetic sensors 30 at time t=1 to T, and acquires the sensor observation value matrix L obtained by integrating these measured values as the sensor observation value vectors l⁽¹⁾ to l^{(T)}.

Next, the magnetic field calculation unit 102 calculates the calculated magnetic field value matrix B from the sensor observation value matrix L, the detected vector matrix K, and the positional information matrix P (step S102). Specifically, the magnetic field calculation unit 102 calculates the calculated magnetic field value matrix B by the following expression (25) obtained by substituting Expression (17) for Expression (7), from the sensor observation value matrix L obtained in step S101 and the detected vector matrix K and the positional information matrix P which are obtained through the calibration process. Meanwhile, the values of the elements of the positional information matrix P are stored in the storage unit 110 in advance, and the values of the elements of the detected vector matrix K are stored in the storage unit 110 in the calibration process.

B=PA=P(KP)⁺L (25)

Finally, the magnetic field calculation unit 102 calculates a magnetic field to be measured by extracting calculated magnetic field values (bᵢₓ⁽¹⁾, b_{iy}⁽¹⁾, b_{iz}⁽¹⁾) to (bᵢₓ^{(T)}, b_{iy}^{(T)}, b_{iz}^{(T)}) at time t=1 to N at the positions of the respective first magnetic sensors 11 from the calculated magnetic field value matrix B calculated in step S102 (step S103), thereby terminating the magnetic field measurement process.

### 1-7. Operational Effects

As described above, in this embodiment, for example, the calibration unit 101 estimates a magnetic field on the basis of a detected vector (detected vector matrix K), positional information (positional information matrix P) of W magnetic sensors (the first magnetic sensors 11 and the second magnetic sensors 30), and measured values (sensor observation value matrix L) of W magnetic sensors by using initial values of detected vectors (detected vector matrix K) of W magnetic sensors as design values (Expression (7) and Expression (17)), and updates the detected vectors (detected vector matrix K) on the basis of the estimated magnetic field (calculated magnetic field value matrix B).

According to the magnetic field measurement apparatus 1 of this embodiment, the calibration unit 101 estimates a magnetic field on the basis of detected vectors, positional information of W magnetic sensors, and measured values of W magnetic sensors by using design values having small differences from true values as initial values of the detected vectors of W magnetic sensors in a state where a magnetic field to be measured is not measured, and thus can update the detected vectors (information regarding the directions and gains of detection axes) of W magnetic sensors to values approximate to the true values with a high level of accuracy. According to the magnetic field measurement apparatus 1 of this embodiment, the magnetic field calculation unit 102 can calculate the magnetic field to be measured with a high level of accuracy in consideration of the directions of the detection axes of W magnetic sensors by using the detected vectors updated with a high level of accuracy.

In addition, in this embodiment, the calibration unit 101 estimates measured values of W magnetic sensors on the basis of the updated detected vectors (detected vector matrix K) and the estimated magnetic field (calculated magnetic field value matrix B) (Expression (7) and Expression (12)), and repeats the process of estimating a magnetic field and the process of updating detected vectors (detected vector matrix K) until norms (the norm ∥V∥ of the observation value error matrix V) of differences between the estimated measured values (calculated observation value matrix L′) of W magnetic sensors and measured values (sensor observation value matrix L) of the magnetic sensors become smaller than a threshold value (allowable value ε).

In this manner, according to the magnetic field measurement apparatus 1 of this embodiment, the calibration unit 101 can converge a detected vector to a value approximate to a true value by repeatedly performing a process of estimating a magnetic field and a process of updating a detected vector. Therefore, according to the magnetic field measurement apparatus 1 of this embodiment, the magnetic field calculation unit 102 can calculate a magnetic field to be measured with a high level of accuracy by using the detected vector approximate to the true value.

In addition, in this embodiment, the calibration unit 101 updates a detected vector kᵢ of the magnetic sensor i for each magnetic sensor i on the basis of a measured value (sensor observation value vector lᵢ) of the magnetic sensor i and an estimated value (calculated magnetic field value matrix bᵢ) of a magnetic field at the position of the magnetic sensor i in the estimated magnetic field (calculated magnetic field value matrix B) (Expression (20)).

In this manner, according to the magnetic field measurement apparatus 1 of this embodiment, the calibration unit 101 can correctly update the detected vectors to values approximate to true values by directly calculating the detected vectors of the magnetic sensors, on the basis of measured values of W magnetic sensors and an estimated value of a magnetic field at the positions of W magnetic sensors, rather than updating the detected vectors by matrix calculation.

In addition, in this embodiment, the calibration unit 101 estimates a magnetic field (calculated magnetic field value matrix B) by approximating the magnetic field by a polynomial expression (Expression (7)) with the positions of W magnetic sensors as variables and calculating the polynomial expression (Expression (7) and Expression (17)) on the basis of detected vectors (detected vector matrix K), positional information (positional information matrix P), and measured values (sensor observation value matrix L) of W magnetic sensors.

In this manner, according to the magnetic field measurement apparatus 1 of this embodiment, the calibration unit 101 can approximate a magnetic field by a polynomial expression with the positions of W magnetic sensors as variables with a high level of accuracy, and thus can estimate the magnetic field with a high level of accuracy by calculating the polynomial expression.

### 2. Second Embodiment

In the magnetic field measurement apparatus 1 (method of calibrating the magnetic field measurement apparatus) according to the first embodiment, the nonlinear polynomial expression (1) showing the distribution of a magnetic field is set without considering the original regularity of the magnetic field at all. On the other hand, a magnetic field measurement apparatus 1 (method of calibrating the magnetic field measurement apparatus) according to a second embodiment is different from that of the first embodiment in that a rule of the divergence of a magnetic field being zero is reflected, and is the same as that of the first embodiment in the other respects. That is, in the magnetic field measurement apparatus 1 (method of calibrating the magnetic field measurement apparatus) according to the second embodiment, it is assumed that the following expression (26) is established. A calibration unit 101 calculates the polynomial expression (1) (specifically, Expression (7)) on the assumption that the divergence of the magnetic field is zero.

$$div \vec{b} = \frac{\partial {b}_{x}}{\partial x} + \frac{\partial {b}_{y}}{\partial y} + \frac{\partial {b}_{z}}{\partial z} = 0 \qquad ( 26 )$$

When a relationship between the coefficients is obtained by substituting Expression (1) for Expression (26), the following expression (27) is established.

aₓ₂+a_{y3}+a_{z4}+2aₓ₈x+a_{y5}x+a_{z7}x+2a_{y9}y+aₓ₅y+a_{z6}y+2a_{z10}z+a_{y6}z+aₓ₇z=(aₓ₂+a_{y3}+a_{z4})+(2aₓ₈+a_{y5}a_{z7})x+(2a_{y9}+aₓ₅+a_{z6})y+(2a_{z10}+a_{y6}+aₓ₇)z=0 (27)

Relational expressions of the following expression (28) are obtained using a fact that Expression (27) is an identity.

aₓ₂+a_{y3}+a_{z4}=0

2aₓ₈+a_{y5}+a_{z7}=0

2a_{y9}+aₓ₅+a_{z6}=0

2a_{z10}+a_{y6}+aₓ₇=0 (28)

Since four relational expressions shown in Expression (28) are obtained, the number of coefficients aₓ₁ to aₓ₁₀, a_{y1} to a_{y10}, and a_{z1} to a_{z10} which is 30 in the first embodiment is reduced to 26. The calibration unit 101 calculates (updates) a detected vector matrix K in accordance with the procedure of FIG. 6. Therefore, according to the magnetic field measurement apparatus 1 (method of calibrating the magnetic field measurement apparatus) of the second embodiment, the amount of calculation of the calibration unit 101 is reduced, or the accuracy of calculation (accuracy of calibration) of the detected vector matrix K is improved.

### 3. Third Embodiment

In the magnetic field measurement apparatus 1 (method of calibrating the magnetic field measurement apparatus) according to the first embodiment, the nonlinear polynomial expression (1) showing the distribution of a magnetic field is set without considering the original regularity of the magnetic field at all. On the other hand, a magnetic field measurement apparatus 1 (method of calibrating the magnetic field measurement apparatus) according to a third embodiment is different from that of the first embodiment in that a rule of the rotation of a magnetic field being zero is reflected, and is the same as that of the first embodiment in the other respects. That is, in the magnetic field measurement apparatus 1 (method of calibrating the magnetic field measurement apparatus) according to the third embodiment, it is assumed that the following expression (29) is established. A calibration unit 101 calculates the polynomial expression (1) (specifically, Expression (7)) on the assumption that the rotation of the magnetic field is zero. Meanwhile, a condition for setting a conduction current and a displacement current to be zero in a space to be measured is necessary in order to set the rotation of the magnetic field to be zero, but it is assumed that the condition is satisfied.

$$rot \vec{b} = ( \frac{\partial {b}_{z}}{\partial y} - \frac{\partial {b}_{y}}{\partial z} ) i + ( \frac{\partial {b}_{x}}{\partial z} - \frac{\partial {b}_{z}}{\partial x} ) j + ( \frac{\partial {b}_{y}}{\partial x} - \frac{\partial {b}_{x}}{\partial y} ) k = 0 \qquad ( 29 )$$

When a relationship between the coefficients is obtained by substituting Expression (1) for Expression (29), the following expression (30) is established.

$$\frac{\partial {b}_{z}}{\partial y} - \frac{\partial {b}_{y}}{\partial z} = {a}_{x 3} - {a}_{y 4} + ( {a}_{z 5} - {a}_{y 7} ) x + ( 2 {a}_{z 9} - {a}_{y 6} ) y + ( {a}_{z 6} - 2 {a}_{y 10} ) z = 0 \\ \frac{\partial {b}_{x}}{\partial z} - \frac{\partial {b}_{z}}{\partial x} = {a}_{x 4} - {a}_{z 2} + ( {a}_{x 7} - 2 {a}_{z 8} ) x + ( {a}_{x 6} - {a}_{z 5} ) y + ( 2 {a}_{x 10} - {a}_{x 7} ) z = 0 \\ \frac{\partial {b}_{y}}{\partial x} - \frac{\partial {b}_{x}}{\partial y} = {a}_{y 2} - {a}_{x 3} + ( 2 {a}_{y 8} - {a}_{x 5} ) x + ( {a}_{y 5} - 2 {a}_{x 9} ) y + ( {a}_{xy7} - {a}_{x 6} ) z = 0 \qquad ( 30 )$$

Relational expressions of the following expression (31) are obtained using a fact that Expression (30) is an identity.

a_{z3}=a_{y4}

a_{z5}=a_{y7}

2a_{z9}=a_{y6}

a_{z6}=2a_{y10}

aₓ₄=a_{z2}

aₓ₇=2a_{z8}

aₓ₆a_{z5}

2aₓ₁₀=a_{z7}

a_{y2}=aₓ₃

2a_{y8}=aₓ₃

a_{y5}=2aₓ₉

a_{y7}=aₓ₆ (31)

Twelve relational expressions shown in Expression (31) are obtained. Here, a_{y7}=aₓ₆ is obtained from a_{z5}=a_{y7} and aₓ₆=a_{z5}. Accordingly, since 11 relational expressions are actually obtained, the number of coefficients aₓ₁ to aₓ₁₀, a_{y1} to a_{y10}, and a_{z1} to a_{z10} which is 30 in the first embodiment is reduced to 19. The calibration unit 101 calculates (updates) a detected vector matrix K in accordance with the procedure of FIG. 6. Therefore, according to the magnetic field measurement apparatus 1 (method of calibrating the magnetic field measurement apparatus) of the third embodiment, the amount of calculation of the calibration unit 101 is reduced, or the accuracy of calculation (accuracy of calibration) of the detected vector matrix K is improved.

Meanwhile, in this embodiment, the calibration unit 101 may further calculate the polynomial expression (1) (specifically, Expression (7)) on the assumption that the divergence of a magnetic field is zero, similar to the second embodiment. Thereby, the number of coefficients is reduced to fifteen due to a further reduction by four, and thus the amount of calculation of the calibration unit 101 is reduced, or the accuracy of calculation (accuracy of calibration) of the detected vector matrix K is further improved.

### 4. MODIFICATION EXAMPLE

The invention is not limited to this embodiment, and various modifications can be made without departing from the scope of the invention.

For example, in the above-described embodiments, the calibration unit 101 of the magnetic field measurement apparatus 1 performs a calibration process, but a calibration apparatus different from the magnetic field measurement apparatus 1 may perform a calibration process of the magnetic field measurement apparatus 1. That is, the processing apparatus 2 of the magnetic field measurement apparatus 1 may not include the calibration unit 101. In this case, the calibration apparatus writes a detected vector matrix K obtained through the calibration process in the storage unit 110 of the processing apparatus 2, and the magnetic field measurement apparatus 1 (magnetic field calculation unit 102) may perform a magnetic field calculation process by using the detected vector matrix K written in the storage unit 110.

In addition, for example, in the above-described embodiments, the second magnetic sensors 30 are provided in order to increase the accuracy of estimation of a magnetic field by using measured values at positions widely dispersed in a calibration process, but the magnetic field measurement apparatus 1 may not include the second magnetic sensors 30. In this case, the calibration unit 101 of the processing apparatus 2 may estimate the magnetic field by using measured values of the first magnetic sensors 11 and may calculate (update) a detected vector matrix K.

In addition, for example, in the above-described embodiments, the magnetic field measurement apparatus 1 measures a heart magnetic field or a brain magnetic field of the test subject 9 (living body), but the magnetic field measurement apparatus 1 may measure a biomagnetic field other than the heart magnetic field or the brain magnetic field, or may measure a magnetic field (week magnetic field) other than the biomagnetic field.

The above-described embodiments and modification example are just examples, and are not limited thereto. For example, the embodiments and the modification example can also be appropriately combined with each other.

The invention includes substantially the same configurations (for example, configurations having the same functions, methods and results, or configurations having the same objects and effects) as the configurations described in the embodiments. In addition, the invention includes a configuration obtained by replacing non-essential portions in the configurations described in the embodiments. In addition, the invention includes a configuration that exhibits the same operational effects as those of the configurations described in the embodiment or a configuration capable of achieving the same objects. In addition, the invention includes a configuration obtained by adding the configurations described in the embodiments to known techniques.

What is claimed is:

1. A magnetic field measurement apparatus comprising:
a plurality of magnetic sensors;
a calibration unit that estimates a magnetic field on the basis of detected vectors of the magnetic sensors, positional information of the magnetic sensors, and measured values of the magnetic sensors, and updates the detected vectors on the basis of the estimated magnetic field; and
a magnetic field calculation unit that calculates a magnetic field to be measured on the basis of the measured values of the magnetic sensors and the detected vectors updated by the calibration unit.

2. The magnetic field measurement apparatus according to claim 1,
wherein the calibration unit estimates the measured values of the magnetic sensors on the basis of the updated detected vectors and the estimated magnetic field, and repeats a process of estimating the magnetic field and a process of updating the detected vectors until norms of differences between the estimated measured values of the magnetic sensors and measured values of the magnetic sensors become smaller than a threshold value.

3. The magnetic field measurement apparatus according to claim 1,
wherein initial values of the detected vectors are design values.

4. The magnetic field measurement apparatus according to claim 1,
wherein the calibration unit updates a detected vector of each of the magnetic sensors, on the basis of a measured value of the magnetic sensor and an estimated value of the magnetic field at a position of the magnetic sensor in the estimated magnetic field.

5. The magnetic field measurement apparatus according to claim 1,
wherein the calibration unit estimates the magnetic field by approximating the magnetic field by a polynomial expression with positions of the magnetic sensors as variables and calculating the polynomial expression on the basis of the detected vectors, the positional information, and the measured values of the magnetic sensors.

6. The magnetic field measurement apparatus according to claim 5,
wherein the calibration unit calculates the polynomial expression on the assumption that divergence of the magnetic field is zero.

7. The magnetic field measurement apparatus according to claim 5,
wherein the calibration unit calculates the polynomial expression on the assumption that rotation of the magnetic field is zero.

8. A method of calibrating a magnetic field measurement apparatus that calculates a magnetic field to be measured on the basis of measured values of a plurality of magnetic sensors and detected vectors of the plurality of magnetic sensors, the method comprising:
acquiring the measured values of the magnetic sensors;
estimating the magnetic field on the basis of the detected vectors, positional information of the magnetic sensors, and the measured values of the magnetic sensors; and
updating the detected vectors on the basis of the estimated magnetic field.
